(19) United States
(12) Patent Application Publication
(10) Pub. No.: US 2024/0138730 A1
(43) Pub. Date: May 2, 2024

(54) Thread-Based Oxygen Sensor

(71) Applicants: Trustees of Tufts College, Medford, MA (US); DTAMedical, Loulle (FR)

(72) Inventors: Sameer Sonkusale, Lincoln, MA (US); Junfei Xia, Allston, MA (US); Francois Dufay, Loulle (FR)

(21) Appl. No.: 18/548,611

(22) PCT Filed: Mar. 4, 2022
(86) PCT No.: PCT/US2022/018860

§ 371 Date: Sep. 1, 2023

Related U.S. Application Data
(60) Provisional application No. 63/156,640, filed on Mar. 4, 2021.

(51) Int. Cl. A61B 5/1473; A61B 5/00; A61B 5/145

(52) U.S. Cl. CPC A61B 5/1473; A61B 5/14542; A61B 5/445

(57) ABSTRACT

An oxygen sensor for sensing dissolved oxygen concentration in tissue includes an electrochemical sensor comprising first and second conductive threads having proximal sections connected to a potentiostat. The first thread forms a cathode, and the second thread forms an anode.

### CROSS-REFERENCE TO RELATED APPLICATIONS

This international application claims priority to U.S. Application Ser. No. 63/156,640, filed on Mar. 4, 2021, the content of which is incorporated herein by reference.

### BACKGROUND

Oxygen plays a key role in human physiology. For example, it is used in energy production through cellular respiration. It also participates in various bodily function ranging from memory storage and cognitive function to tissue repair and regeneration during would healing. A lack of oxygen in tissue or blood is associated with many diseases, such as cerebral ischemia, congestive heart failure, and cancer.

In addition, oxygen is abnormally modulated in various diseases. As a result, in situ monitoring of oxygen is an important biomarker for assessing insufficient oxygen delivery and the resulting tissue damage.

A lack of oxygen is of particular importance in the context of wound healing. The process of healing a would is characterized by the stepwise inflammation, proliferation, and remodeling phases. Disruption of these phases, for example by lack of oxygen, can lead to a chronic wound. The level of tissue oxygenation is thus an important biomarker in the process of wound healing.

Known oxygen sensors only measure superficial oxygen levels in a shallow wound bed. However, these oxygen levels, even if accurately determined, will differ from that present deep in the tissue. This is because oxygen at or near the surface equilibrates with atmosphere oxygen levels.

A difficulty that arises in known oxygen sensors is that it is difficult to measure oxygen levels deep inside tissue. This can occur as a result of a mismatch between the electrode and the tissue at the interface between the two structures.

Known oxygen sensors include those that rely on interaction between dissolved oxygen and electromagnetic waves, particularly in the near infrared. However, electromagnetic waves tend to attenuate when passing through bodily fluids. As a result, it is difficult to use them to measure dissolved oxygen concentrations deep in the body.

### SUMMARY

In one aspect, the invention features an apparatus for sensing dissolved oxygen concentration in tissue. Such an apparatus includes an oxygen sensor having an electrochemical sensor that comprises first and second conductive threads having proximal sections connected to a potentiostat. The first thread forms a cathode; the second thread forms an anode.

Embodiments include those in which one or more of the threads includes silver, those in which one or more of the threads include gold, and those in which one or more of the threads include platinum.

In some embodiments, the second thread includes a bare distal section, and a shielded section between the proximal and distal sections. Among these are embodiments in which the electrochemical sensor is a wire-type sensor having an extended sensing zone and the first thread includes a bare distal section and a shielded section between the proximal and distal sections. Also among these embodiments are those in which electrochemical sensor is a tip-type sensor and the first thread includes a shielded distal section having an exposed distal tip that defines a distal sensing zone.

Some embodiments include hydrogel in contact with one or more of the threads.

In some embodiments, one or more of the threads comprises a wire.

Embodiments further include those in which the electrochemical sensor comprises a distal tip that defines a distal sensing zone, hydrogel at the distal tip, and an oxygen-permeable membrane disposed over the hydrogel.

Other embodiments include a pad. In such embodiments, the electrochemical sensor is a wire-type sensor having an extended sensing zone that is disposed on the pad.

In still other embodiments, the electrochemical sensor is a wire-type sensor having an extended sensing zone that is covered with hydrogel.

### BRIEF DESCRIPTION OF THE FIGURES

FIG. 1 shows a wire-type oxygen sensor; and

FIG. 2 shows a tip-type oxygen sensor;

FIG. 3 shows multiple tip-type sensors deployed in a wound bed;

FIG. 4 shows steps in the manufacture of an electrochemical sensor;

FIG. 5 shows a hydrogel-coated wire sensor on a pad; and

FIGS. 6 and 7 show wire sensors bent into shapes to conform to wound geometry.

### DETAILED DESCRIPTION

As a result of its ability to conform to the human body, its mechanical stability, and its low cost, a suitably modified thread is useful has a point-of-care biosensor. Advantages of thread-based sensors include the ability to manufacture than without a cleanroom, the large surface area available to interface with an analyte, and its ability to wick.

Unlike rigid metal electrodes, a thread-based electrode is easily inserted inside tissue using only a suture needle. This permits measurements deep in the tissue.

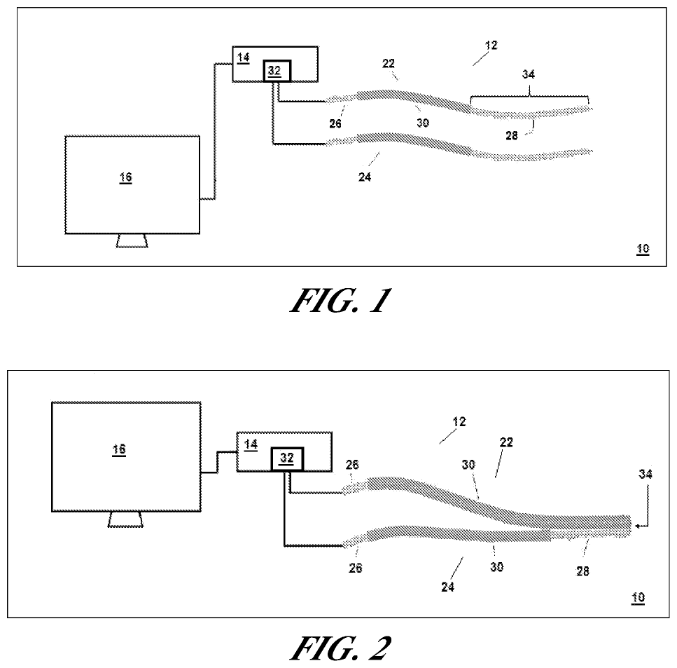

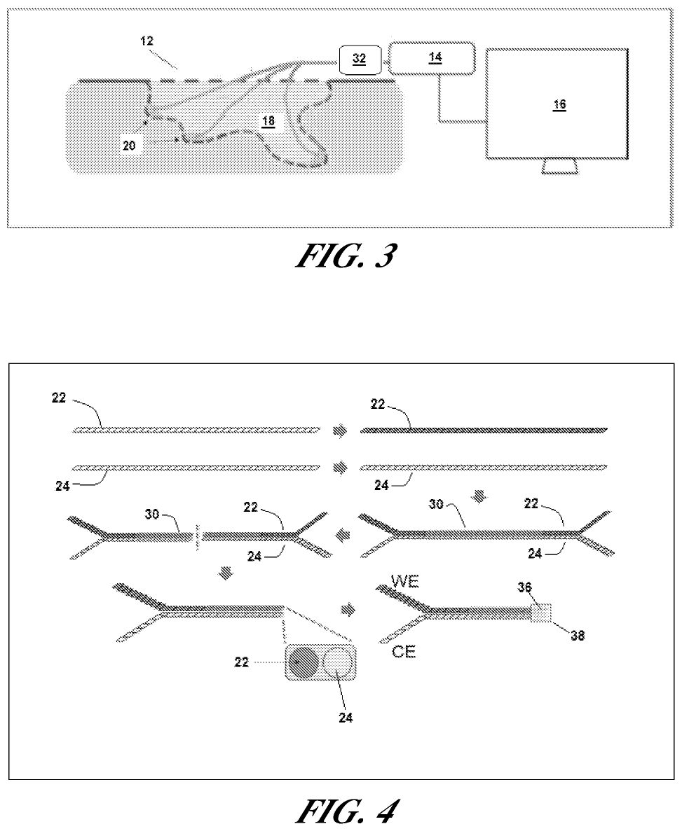

FIGS. 1 and 2 shows two embodiments of an oxygen sensor 10 that relies on threads that have been processed to form thread-based electrochemical sensors. Such sensors are useful for monitoring concentration of dissolved gas, and in particular, dissolved oxygen. The oxygen sensor 10 includes an electrochemical sensor 12 that is connected to read-out electronics 14 connected to a display 16.

FIGS. 1 and 2 show different types of electrochemical sensor 12.

The electrochemical sensor 12 shown in FIG. 1 is a wire-type sensor that is useful for measuring an average oxygenation level across an extended region, such as across a wound bed 18, such as that shown in FIG. 3. That shown in FIG. 2 is a tip-type sensor that is particularly useful for pinpointing a region-of-interest 20 within the wound bed 18.

Referring to FIG. 1, the wire-type sensor 12 comprises first and second threads 22, 24, each of which incorporates a conductive material. A suitable choice of conductive material is a conducting metal that is also biocompatible. Suitable metals include silver, gold, and platinum.

Each thread 22, 24 comprises a bare proximal section 26 and a bare distal section 28 separated by a shielded section 30 that is shielded by a dielectric jacket. Each thread connects to a potentiostat 32 at its proximal section 26. As a result, the first thread 22 serves as a cathode and the second thread 24 serves as an anode. The combination thus forms an electrochemical sensor having a sensing zone 34 that extends along the distal sections 20 of the threads.

In the alternative embodiment shown in FIG. 2, an electrochemical sensor 12 implemented as a tip-type sensor comprises first and second threads 22, 24, each of which incorporates a conductive material. A suitable choice of conductive material is a conducting metal that is also biocompatible. Suitable metals include silver, gold, and platinum.

The first thread 22 comprises a bare proximal section 26. The shielded section 30 covers all but the distal tip of the distal section 28, thus leaving only the distal tip as a sensing zone 34. The second thread 24 has a bare proximal section 26 and a distal section 28 separated by a shielded section 30 that is shorter than that of the first thread 24, thus leaving a larger exposed section.

The proximal sections 26 of the first and second threads 22, 24 connect to corresponding terminals of the potentiostat 32. As a result, the first thread 22 serves as a cathode and the second thread 24 serves as an anode. The combination thus forms an electrochemical sensor having a sensing zone 30 confined to the distal tip of the first thread 22.

In the foregoing embodiments, the cathode is a noble metal that is stable and that does not participate in electrode reactions. Examples of suitable materials include silver and gold. In some embodiments, the anode is a silver-coated thread, and the cathode is a gold wire.

In some embodiments, during operation, the exposed surfaces are covered by a material that suppresses the tendency for fouling during extended use. Suitable materials include a hydrogel.

In operation, the potentiostat 32 provides a steady voltage that drives a current. The current's magnitude is indicative of dissolved oxygen concentration.

In general, there are two major types of electrochemical oxygen sensors: galvanic and polarographic. In a galvanic sensor, two dissimilar metals are paired as anode and cathode. When connected, an output voltage or current is spontaneously generated in response to change in oxygen level. In a polarographic sensor, an external voltage source is applied to both electrodes and the output current is measured. The use of threads as described herein is applicable to both types.

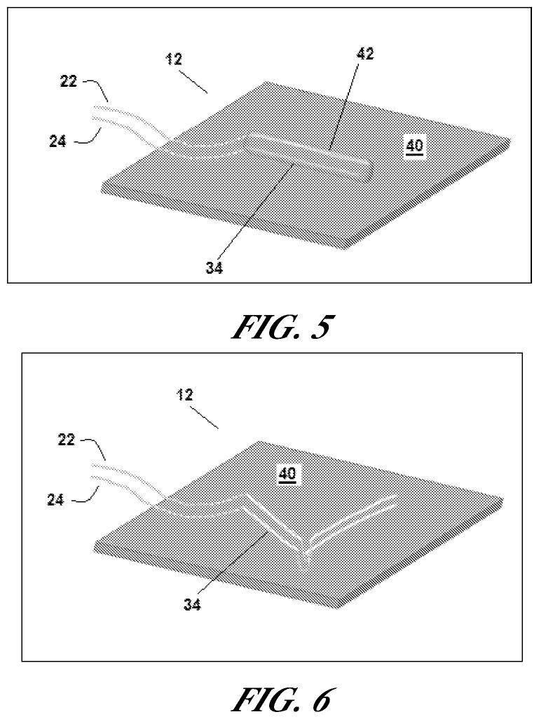

Referring to FIG. 4, a suitable method of making an oxygen sensor 10 includes coating a first thread 22 with carbon ink, coating a second thread 24 with silver or gold ink, and bundling them together with a dielectric ink to form a shielded section 30. In some embodiments, the first and second threads 22, 24 comprise polyester. The threads are then sliced to expose cross-sections thereof.

Zinc is then electrochemically deposited on a cross-section of the first thread 22. This becomes a working electrode WE. The second thread's end becomes the counter electrode CE.

The tips of the threads 22, 24 are then coated with an electrolyte gel 36 and sealed with a protective oxygen-permeable membrane 38. In the illustrated embodiment, the membrane 38 is PDMS. A suitable electrolyte gel 36 is 0.1 mM KOH in agarose.

Although it is permeable to oxygen, the membrane 38 hinders oxygen diffusion to some extent, thus lowering temporal resolution. The temporal resolution can thus be controlled by controlling the membrane's thickness.

To carry out electrochemical measurements, the sensor 12 is connected to a potentiostat 32, which carries out chronoamperometry at a polarographic voltage of 0.7 volts.

An impediment to the use of a thread 22, 24 as an electrode arises from the inherently non-planar surface at the thread's tip and from the thread's natural wicking properties. The coating of electrolyte gel 36 and the sealing with PDMS 38 co-operate to ameliorate these difficulties. Moreover, because the electrode area is small, the overall output current is small, thus slowing consumption of zinc. In addition, being a polarographic sensor, no current is generated when the sensor 12 is turned off. These properties promote a long-lived sensor 12 that can be used for extended periods to monitor oxygen in a chronic wound.

In some embodiments, rather than carrying out electrochemical deposition of zinc, it is useful to provide a commercially-available silver-coated thread. As silver is not an active metal, it is necessary to apply an external voltage to trigger the electrode reaction, in which silver is dissolved at the anode and oxygen reacts with water at the cathode. In preferred embodiments, the cathode is a noble metal that is stable and that does not participate in electrode reactions. In the following two types of sensor, gold and silver are selected as cathode materials, respectively.

Some embodiments of a flexible, thread-based electrochemical sensor along the lines of the foregoing respond to change in dissolved oxygen concentrations and does so linearly within a range of 2.94-47.86 milligrams per liter of dissolved oxygen with a sensitivity of 624.17 nanoamperes per milligram-liter of dissolved oxygen. The sensor shows good repeatability with data variation less than 2%. The sensor also displays relatively good stability over the period of one week in a simulated tissue environment. Such a sensor can be easily placed and secured in the wound bed using non-toxic PDMS as a supporting material.

In some embodiments, the sensor 12 comprises silver-coated first and second threads 22, 24 that have been cleaned by isopropanol and cut into ten-centimeter segments, of which two centimeters is used as a sensing zone 34 and fixed onto a thin PDMS pad 40, as shown in FIG. 5. The remaining segments are pulled to the other side of the pad using a needle. The connection point is then sealed with dielectric ink. The sensing zone 34 is then coated with a gel layer 42. The gel layer 42 ensures the presence of electrolytes for the electrode reactions. A suitable material for the gel layer 42 is hydrogel.

The sensor 12 can be easily applied to a wound bed 18 with the gel layer 42 facing inward. Such design allows easy connection to a readout electronics 14. The transparency of the PDMS pad 40 and gel layer 42 makes it possible to continue to visually monitor the wound bed 18. The length of the sensing zone 34 can be freely adjusted depending on the area of the wound bed 18.

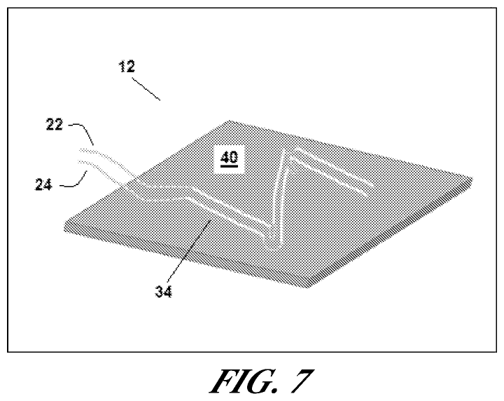

The shape of the sensing zone 34 can be adjusted as well due to the flexible nature of the thread. For example, FIG. 6 shows an L-shaped sensing zone 34 and FIG. 7 shows a Z-shaped sensing zone 34.

As is apparent from FIGS. 7 and 8, the first and second threads 22, 24 can be placed into the wound bed 18 without the gel layer 42 provided that the tissue itself provides sufficient concentration of ions, particularly chloride ions, for the reaction.

However, a gel layer 42 has additional advantages. These include protecting the sensing zone 34 from mechanical abrasion, such as that caused by displacement. In addition, the gel layer 42 provides protection from toxicity, if any, induced by the thread 22, 24 or the electrode reactions. Although a gel layer 42 inevitably hinders oxygen diffusion, it does not do so to an appreciable extent.

In some embodiments, it is useful to take measures to avoid having the measured oxygen concentration be affected by oxygen in the atmosphere that may diffuse through the PDMS pad 40. Such measures include embedding the sensor 12 inside the tissue or coating the PDMS pad 40 with an oxygen-diffusion barrier, examples of which include PPC and PVA membrane.

The principles described herein can also be used to sense other analytes, such as glucose, or to sense pH or pOH. A glucose sensor is implemented by implemented by immobilizing glucose oxidase enzyme at the sensing zone 34 using a matrix, such as chitosan or another polymer. A pH sensor is implemented by coating the sensing zone 34 with polyaniline .on the conductive tip.

Other embodiments feature those in which the sensing zone 34 has been functionalized to detect other ions, such as sodium ions and potassium ions, or other analytes, such as lactate. In still other embodiments, the sensing zone 34 is functionalized with antibodies or aptamers for detection of target biomolecules, including nucleic acids, such as DNA and RNA, and proteins and amino acids.

1-16. (canceled)

17. An apparatus comprising an oxygen sensor for sensing dissolved oxygen concentration in tissue, said oxygen sensor comprising an electrochemical sensor comprising first and second threads having proximal sections connected to a potentiostat, at least one of said threads being a thread that wicks liquid in which said oxygen is dissolved, said first thread forming a cathode of said electrochemical sensor and said second thread forming an anode of said electrochemical sensor.

18. The apparatus of claim 17, wherein at least one of said threads comprises a material selected from the group consisting of silver and platinum.

19. The apparatus of claim 17, wherein said electrochemical sensor is configured such that no current is generated when said electrochemical sensor has been turned off.

20. The apparatus of claim 17, wherein at least one of said threads comprises an extended sensing zone that is covered by hydrogel.

21. The apparatus of claim 17, wherein said first thread has a distal section, wherein said second thread has a distal section, wherein said distal section of said first thread is shielded except for a distal tip of said distal section of said first thread, wherein said distal section of said second thread is bare, wherein said distal tip of said distal section of said first thread is bare, and wherein said electrochemical sensor has a sensing zone that is confined to said distal tip of said first thread.

22. The apparatus of claim 17, wherein said first thread comprises a distal section and a shielded section, wherein said shielded section of said first thread is between said proximal section of said first thread and said distal section of said first thread, wherein said distal section of said first thread is a bare distal section, wherein said second thread comprises a distal section and a shielded section, wherein said shielded section of said second thread is between said proximal section of said second thread and said distal section of said second thread, and wherein said distal section of said second thread is a bare distal section,

23. The apparatus of claim 17, wherein said electrochemical sensor is a tip-type sensor that has a sensing zone that is confined to a tip of said first thread.

24. The apparatus of claim 17, further comprising hydrogel that is in contact with at least one of said threads.

25. The apparatus of claim 17, wherein at least one of said threads incorporates a conductive material therein.

26. The apparatus of claim 17, wherein said electrochemical sensor is a wire-type sensor having a sensing zone that extends along a section of said first thread.

27. The apparatus of claim 17, wherein said electrochemical sensor is configured to measure an oxygenation level in a wound bed.

28. The apparatus of claim 17, wherein said electrochemical sensor comprises a galvanic sensor.

29. The apparatus of claim 17, wherein said electrochemical sensor comprises a polarographic sensor.

30. The apparatus of claim 17, wherein said electrochemical sensor comprises a distal tip that defines a distal sensing zone, hydrogel at said distal tip, and an oxygen-permeable membrane disposed over said hydrogel.

31. The apparatus of claim 17, further comprising a pad, wherein one of said first and second threads has an extended sensing zone that is disposed on said pad.

32. The apparatus of claim 17, wherein said electrochemical sensor is configured to measure an average oxygenation level across a sensing zone thereof.

33. The apparatus of claim 17, wherein at least one of said threads comprises polyester.

34. The apparatus of claim 17, wherein said threads are adjustable to transition between an L-shaped sensing zone and a Z-shaped sensing zone.

35. The apparatus of claim 17, wherein said first thread is coated with carbon ink, wherein said first and second threads are bundled together with a dielectric ink to form a shielded section, wherein zinc is electrochemically deposited on a cross section of said first thread, and wherein tips of said first and second threads are coated with an electrolyte gel and sealed with a protective oxygen-permeable membrane.

36. An apparatus comprising an oxygen sensor for sensing dissolved oxygen concentration in tissue, said oxygen sensor comprising an anode and a cathode, wherein said anode comprises a thread that wicks liquid that contains said dissolved oxygen, wherein said cathode comprises a wire, and wherein said thread and said wire having distal sections that connect to a potentiostat.
